(19) United States
(12) Patent Application Publication
(10) Pub. No.: US 2010/0330170 A1
(43) Pub. Date: Dec. 30, 2010

(54) Flavored Vegetarian Cellulose Capsule and Methods for Producing said Capsule

(75) Inventor: Jonathan Gilinski, Miami, FL (US)

Correspondence Address: ROBERT M. SCHWARTZ, P.A., P.O. BOX 221470, HOLLYWOOD, FL, 33022, US

(73) Assignee: Capsuline, Inc., Pompano Beach, FL (US)

(21) Appl. No.: 12/877,256

(22) Filed: Sep. 8, 2010

Related U.S. Application Data
(63) Continuation of application No. 10/907,315, filed on Mar. 29, 2005.

(51) Int. Cl. A61K 9/48; A23L 1/0534; A23L 1/302; A23L 1/303

(52) U.S. Cl. 424/452; 426/98; 426/72; 426/73; 426/61; 426/231

(57) ABSTRACT

A flavored vegetable starch capsule and the method of manufacture of the flavored capsule is provided. The capsule may comprise (a) from about 95% to about 100% parts by weight of cellulose, such as hydroxymethylcellulose; (b) from about 0.5% to about 5.5% by weight of a suitable hydrogenated saccharide, such as sorbitol; (c) from about 0.2 to about 2.5% of a lubricant, such as silicon dioxide; (d) up to about 10% purified water; to which is added about 1/10 parts by weight of liquid flavoring.

This application is a continuation of U.S. patent application Ser. No. 10/907,315, filed Mar. 29, 2005 which claims priority based on U.S. Provisional Patent No. 60/517,524 filed on Oct. 8, 2004, entitled Process for the Manufacture of a Flavored Gelatin Cap, unassigned, the disclosures of which are incorporated herein by reference in their entirety.

### FIELD OF THE INVENTION

This invention relates to vegetarian cellulose and methods of manufacture of capsules, and more specifically the introduction of vegetarian cellulose capsules that are comprised of over 95% cellulose by dry weight to which is added containing 2% or more of liquid flavoring, and only minimal amounts of hydrogenated saccharide and lubricant.

### BACKGROUND OF INVENTION

The taste of many medicinal and nutritive components can be quite distinctive and potentially unpleasant. Improvements in the taste of certain drugs and nutritional supplements can lead to a higher compliance by consumers. A higher compliance will result in greater commercial success for the drug and supplement manufacture and in increased health and well being particular consumers.

Taste is both a matter of purely subjective preference. Yet human taste is also strongly influenced by experience and cultural impressions. Broad generalizations about consumer taste presence can thus sometimes be relied upon in predicting market acceptance of specific drug and nutritive formulations. Numerous supplements and drugs are known for bad taste or smell, such as alpha-lipoic acid, N-acetyl cysteine, omega-3 fatty acids, and choline.

Attempts to create a flavored capsule have been limited by the requirement to use high amounts of softeners, including hydrogenated saccharide, synonymous with polyol (polyalcohol) or gelatin softeners, and consequently lower amounts of flavoring. U.S. Pat. No. 6,652,879, to Opheim, teaches a flavored gelatin capsule in which the concentration of flavoring is in the range between about 0.25% and about 1.5% of the gelatin capsule, and the capsule contains a gelatin softener in a concentration of 10% to 35% of the capsule. U.S. Pat. No. 6,641,837, also to Opheim, teaches a flavored vegetable starch capsule in which the concentration of flavoring is in the range between about 0.25% and about 1.5% of said flavored vegetable starch capsule, and wherein one polyol, such as glycerol or sorbitol, is present in the range of between 10% and 35% of said flavored vegetable starch capsule. U.S. Pat. Nos. 6,475,542 and 6,592,916, to Soeda, teaches an edible hydrophobic substance; comprised of single cored microcapsules formed by salting-out of a combination of gelatin and an edible salt, to obtain an unhardened capsule wall, followed by treating said unhardened capsule wall with transglutaminase as a crosslinking agent for hardening the capsule wall. The method is suitable to prepare powder particles having a particle size of not larger than 100 .mu.m, preferably the order of several tens of microns. U.S. Pat. No. 5,264,223, to Yamamoto, describes the inclusion of a flavor in a capsule, but does not specify the concentration that can be provided by its teachings.

U.S. Pat. No. 5,709,895, to Tanaka, teaches a process for producing flavor-containing sugar-free capsules with a content of flavor of up to 20%, comprising the steps of a) heating a carbohydrate mixture containing at least one modified starch and at least one hydrogenated saccharide at a weight ratio of from about 15:85 to about 85:15 on a solid basis to give a molten material; (b) adding a flavor to said molten material and mixing said flavor and said molten material to thereby give a uniform mixture; (c) solidifying said uniform mixture by rapidly cooling under extrusion to yield a solid material; and (d) cutting or grinding the solid material thus obtained, wherein said at least one hydrogenated saccharide is one or more substances selected from the group consisting of xylitol, lactitol, maltitol, isomalt, and hydrogenated corn syrup. Like Opheim, a higher amount of hydrogenated saccharide, 15% on a solid basis, is required. Moreover, a cutting and grinding of the solid material obtained is necessary that this inventor has learned to avoid. Tanaka, while including sorbitol among they hydrogenated saccharides that may be used, teaches away from its use in stating that the production of flavor-containing capsules using sugar alcohols such as sorbitol requires “troublesome operations” for example, heating at a high temperature, and that the capsules thus obtained have a high hygroscopicity caused by the hygroscopicity of sugar alcohols as the main component and thus can be hardly stored for a long period of time. This inventor, by reducing the amount of hydrogenated saccharides to below 5.5% of the dry weight of the solution, avoids the difficulties described by Tanaka.

### SUMMARY OF THE INVENTION

This inventor has discovered a method and composition providing a flavored vegetarian cellulose capsule suitable for encapsulating a dose, in which a flavoring solution is present in a concentration which is in the range between about 2% and about 20% of the total weight of said vegetarian cellulose capsule.

In its most specific embodiment, the flavoring solution is present in an amount of about 10% of the total weight. The flavored vegetarian cellulose capsule of the invention may be used to encapsulate a dose selected from the group consisting of nutritional supplements, over-the-counter drugs, and prescription drugs. The flavoring solution may be chosen from the group consisting of strawberry, grape, mint, orange, blueberry, chocolate, cocoa, lemon, nut, almond, cashew, macadamia nut, coconut, blueberry, blackberry, raspberry, peach, lemon, lime, banana, chili pepper, pepper, cinnamon, and pineapple. Most specifically, it is mint, orange strawberry or grape.

### DETAILED DESCRIPTION OF THE INVENTION

This inventor has discovered a method and composition providing a flavored vegetarian cellulose capsule suitable for encapsulating a dose, said capsule comprised of a cellulose, present in an amount ranging from about 95% to about 98% by dry weight, a hyrogenated saccharide, present in an amount ranging from about 0.5% to about 5.5% by dry weight, a lubricant, present in an amount ranging from about 0.2% to about 2.5% by dry weight; purified water present in an amount ranging from about 1:10 to about 1:20 of the dry weight of the capsule and a flavoring solution, wherein said flavoring solution is present in said flavored cellulose capsule in a concentration which is in the range between about 2% and about 20% of the total weight of said vegetarian cellulose capsule.

The flavored vegetarian cellulose capsule of the invention may be used to encapsulate a dose selected from the group consisting of nutritional supplements, over-the-counter drugs, and prescription drugs. Any nutritional supplement may be so encapsulated, including but not limited to an amino acid, vitamin, or animal product selected from the group consisting of acetyl-l-carnosine, alpha lipoic acid, amylase, androstendiol, androstendione, arginine, ascorbic acid, B vitamin, beta-carotene, biotin, bromelain, calcium, chicken collagen, chitosan, choline, chondroitin, coenzyme Q10, creatine, dehydroepiandrosterone, diethylmethylaminoethanol, dihydroepiandsterone, dimethylglycine, DMSO, gammahydroxybutric acid (GABA), glucosamine, glutamine, glutathione, hyaluronic acid, hydroxytryptophan, indium, isoleucine, 1-carnitine, lactoferrin, lecithin, leucine, lipase, lumbrokinase, lutein, magnesium, melatonin, Methylcobalamin, methylsulfonylmethane, MGN 3, ornithine, pancreatin, panthethoic acid, papain, para-amino benzoic acid (PABA), phenylalanine, phosphatidylcholine, phosphatidylserine, potassium, pregnenalone, protease, retinoic acid, retinol, s-adenosyl-methionine , selenium, taurine, theanine, thymase, tocopherol, trimethylglycine, tryptophan, tyrosine, valine, vinpocetine, vitamin D, vitamin A, zeathanthine, zinc.; and their nutraceutically acceptable salts, ethers, esters, acid, or other derivatives.

A nutritional supplement may also be chosen from herbal preparations, such as the leaf, root, or extract of a plant selected from the group consisting of artichoke, bilberry, bioflavonoid, boswella, bupleurium, chamomile, chlorophyll, cranberry, damiana, echinacea, essiac, garcinia cambogia, garlic, germanium, ginger, gingko, ginseng, goldenseal, grape seed, green tea, hawthorne berry, hesperidin, hops, horse chestnut hydrangea, hypericum, indole-3-carbinol, licorice, lycopene, nettle root, peppermint, periwinkle, policosanol, psyllium, pygeum, quercetin, raspberry, resveratol, rutin, sassafras, saw palmetto, silymarin, tribulus terestris, turmeric, valerian, wild yam; and their nutraceutically acceptable salts, ethers, esters, acid, or other derivatives.

In its most specific embodiment, the flavoring solution is present in an amount of about 10% of the total weight. The flavoring solution may be chosen from the group consisting of strawberry, grape, mint, orange, blueberry, chocolate, cocoa, lemon, nut, almond, cashew, macadamia nut, coconut, blueberry, blackberry, raspberry, peach, lemon, lime, banana, chili pepper, pepper, cinnamon, and pineapple. Flavoring solutions are sold by manufacturers well known to those of ordinary skill in the art including extracts offered by Watkins Products of Minneapolis, Minn.

The cellulose used may be any suitable cellulose well known to those of ordinary skill in the art including polyacrylates, polymethacrylates, polyvinylpyrrolidone, poly(vinyl acetate), various starches, corn products such as amaizo, amylose and zein, pectin, alkoxylated celluloses, polyesters, polyethers, polyethylene glycol, proteins, nucleic acids, albumin, gelatin, starch, collagen, dextran and modified dextrans, polysaccharides, polylactide/polyglycolide, polyalkylcyanoacrylates, polyacrylamide, polysorbates, polyethylene ethers, polyethylene esters, polyoxyethylene/polyoxypropylene block polymerss, cellulose acetophthalate, hydroxypropylmethyl cellulose, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, methyl cellulose, ethyl cellulose, hydroxyethyl cellulose, propyl cellulose, hydroxypropyl cellulose, lower-substituted hydroxypropyl cellulose, carboxymethyl cellulose, and hydroxypropylmethyl cellulose.

These may also be purchased from manufacturers such as Polymer Laboratories, Inc., of Amherst, Mass. Heterene, Inc., of Paterson, N.J., Klucel.RTM. from Nippon Soda, Japan and Dow Chemical Company, S.A. and others known to those skilled in the art. Most specifically, the cellulose is hydroxypropylmethyl cellulose, also known as hypromellose.

The hydrogenated saccharide may be sorbitol, mannitol, allitol, galactitol, L-mannitol, altritol, L-glucitol, iditol, and hexitol. Most specifically it is sorbitol.

The lubricant used may be silicon dioxide, titanium dioxide, calcium phosphate, calcium sulfate , sodium laurel sulfate, silicified microcrystalline cellulose, magnesium stearate, and talc; most specifically silicon dioxide. Various silicones are available from manufacturers known to those skilled in the art, such as United Chemical Technologies, Inc., of Bristol, Pa. and other suitable lubricants are available from numerous manufacturers including Bioclean Impex of India.

To prepare the flavored vegetarian cellulose capsules several steps are employed:

(a) a flavorless vegetarian cellulose composition is prepared as follows: (1) the primary ingredient, cellulose, is weighed (or the weight verified) to an amount ranging from about 95% to about 98% of the dry weight of a composition; (2) the cellulose is added to a water, at about 10 parts cellulose to 1 part water, in stainless steel jacketed melt tanks. There are written procedures available for the weighing and manufacture of HPMC solution, and the sanitation of the equipment used; (3) a hydrogenated saccharide is weighed to about 0.5 to about 5.5% of the dry weight of the composition; (4) a lubricant is weighed to about 0.2% to about 2.5% of the dry weight of the composition; (5) the hydrogenated saccharide and lubricant are added to the cellulose and water in said stainless steel jacketed melt tanks;
(b) a flavoring solution is prepared with food-grade flavor powder and cold water, or alternatively a flavored liquid base is weighed, to about 1 part flavoring to 49 parts vegetarian cellulose solution to about 1 part flavoring to 4 parts vegetarian cellulose solution by liquid weight;
(c) the flavoring solution or liquid base is added to the flavorless vegetarian cellulose solution;
(d) the flavored vegetarian cellulose solution is melted and de-bubbled at a temperature of about 10 degrees centigrade to about 20 degrees centigrade;
(e) additional water and lubricant are added to achieve a specific gravity of about 1 gm/cm.sup.3.
(f) the flavored vegetarian cellulose solution is transported to the machine that produces vegetable capsules;
(g) hot molds, specifically pins, are dipped into the solution to form the capsule parts of cap and body;
(h) after the flavored vegetarian cellulose solution solution is adhered to the mold and submitted to a drying process with heat and dry air.
(i) the formed parts (cap and body) are stripped from the molds using additional lubricant;
(j) the cap and body are joined.

Optionally, a dye solution is also prepared by weighing dye powders and dissolving them in hot water and the dye solution is added to the flavored vegetarian cellulose solution in the stainless steel tanks before it is melted and de-bubbled.

Any one of numerous available flavors may be used, such as strawberry, grape, mint, orange, blueberry, chocolate, cocoa, lemon, nut, almond, cashew, macadamia nut, coconut, blueberry, blackberry, raspberry, peach, lemon, lime, banana, chili pepper, pepper, cinnamon, and pineapple. Most specifically, it is mint, orange, strawberry, or grape.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

I claim:

1. A flavored vegetarian cellulose capsule suitable for encapsulating a dose, said capsule comprised of a cellulose, present in an amount ranging from about 95% to about 98% by dry weight, a hyrogenated saccharide, present in an amount ranging from about 0.5% to about 5.5% by dry weight, a lubricant, present in an amount ranging from about 0.2% to about 2.5% by dry weight; purified water present in an amount ranging from about 1:10 to about 1:20 of the dry weight of the capsule and a flavoring solution, wherein said flavoring solution is present in said flavored cellulose capsule in a concentration which is in the range between about 2% and about 20% of the total weight of said vegetarian cellulose capsule.

2. The flavored vegetarian cellulose capsule of claim 1 further comprising and encapsulated dose selected from the group consisting of nutritional supplements, over-the-counter drugs, and prescription drugs.

3. The flavored vegetarian cellulose capsule of claim 2 wherein said nutritional supplement is selected from the group consisting of one or more of the group consisting of an amino acid, vitamin, herbal, or animal product selected from the group consisting of acetyl-l-carnosine, alpha lipoic acid, amylase, androstendiol, androstendione, arginine, ascorbic acid, B vitamin, beta-carotene, biotin, bromelain, calcium, chicken collagen, chitosan, choline, chondroitin, coenzyme Q10, creatine, dehydroepiandrosterone, diethylmethylaminoethanol, dihydroepiandsterone, dimethylglycine, DMSO, gammahydroxybutric acid (GABA), glucosamine, glutamine, glutathione, hyaluronic acid, hydroxytryptophan, indium, isoleucine, 1-carnitine, lactoferrin, lecithin, leucine, lipase, lumbrokinase, lutein, magnesium, melatonin, Methylcobalamin, methylsulfonylmethane, MGN 3, ornithine, pancreatin, panthethoic acid, papain, para-amino benzoic acid (PABA), phenylalanine, phosphatidylcholine, phosphatidylserine, potassium, pregnenalone, protease, retinoic acid, retinol, s-adenosyl-methionine , selenium, taurine, theanine, thymase, tocopherol, trimethylglycine, tryptophan, tyrosine, valine, vinpocetine, vitamin D, vitamin A, zeathanthine, zinc. and the leaf, root, or extract of artichoke, bilberry, bioflavonoid, boswella, bupleurium, chamomile, chlorophyll, cranberry, damiana, echinacea, essiac, garcinia cambogia, garlic, germanium, ginger, gingko, ginseng, goldenseal, grape seed, green tea, hawthorne berry, hesperidin, hops, horse chestnut hydrangea, hypericum, indole-3-carbinol, licorice, lycopene, nettle root, peppermint, periwinkle, policosanol, psyllium, pygeum, quercetin, raspberry, resveratol, rutin, sassafras, saw palmetto, silymarin, tribulus terestris, turmeric, valerian, wild yam; and their nutraceutically acceptable salts, ethers, esters, acids, or other derivatives.

4. The flavored vegetarian cellulose capsule of claim 1 wherein the flavoring solution is present in an amount of about 10% of the total weight.

5. The flavored vegetarian cellulose capsule of claim 1 wherein the flavoring solution comprises a flavor chosen from the group consisting of strawberry, grape, mint, orange, blueberry, chocolate, cocoa, lemon, nut, almond, cashew, macadamia nut, coconut, blueberry, blackberry, raspberry, peach, lemon, lime, banana, chili pepper, pepper, cinnamon, and pineapple.

6. The flavored vegetarian cellulose capsule of claim 1 wherein said cellulose comprises a cellulose selected from the group consisting of polyacrylates, polymethacrylates, polyvinylpyrrolidone, poly(vinyl acetate), various starches, corn products such as amaizo, amylose and zein, pectin, alkoxylated celluloses, polyesters, polyethers, polyethylene glycol, proteins, nucleic acids, albumin, gelatin, starch, collagen, dextran and modified dextrans, polysaccharides, polylactide/polyglycolide, polyalkylcyanoacrylates, polyacrylamide, polysorbates, polyethylene ethers, polyethylene esters, polyoxyethylene/polyoxypropylene block polymerss, cellulose acetophthalate, hydroxypropylmethyl cellulose, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, methyl cellulose, ethyl cellulose, hydroxyethyl cellulose, propyl cellulose, hydroxypropyl cellulose, lower-substituted hydroxypropyl cellulose, carboxymethyl cellulose, and hydroxypropylmethyl cellulose.

7. The flavored vegetarian cellulose capsule of claim 1 wherein said cellulose is hydroxypropylmethyl cellulose.

8. The flavored vegetarian cellulose capsule of claim 1 wherein said hydrogenated saccharide is selected from the group consisting of sorbitol, mannitol, allitol, galactitol, L-mannitol, altritol, L-glucitol, iditol, and hexitol.

9. The flavored vegetarian cellulose capsule of claim 7 wherein said hydrogenated saccharide is sorbitol.

10. The flavored vegetarian cellulose capsule of claim 1 wherein said lubricant is selected from the group consisting of silicon dioxide, titanium dioxide, calcium phosphate, calcium sulfate , sodium laurel sulfate, silicified microcrystalline cellulose, magnesium stearate, and talc.

11. The flavored vegetarian cellulose capsule of claim 9 wherein said lubricant is silicon dioxide.

12. A method for manufacturing the flavored vegetarian cellulose capsule comprising the steps of: (a) a flavorless vegetarian cellulose composition is prepared as follows: (1) the primary ingredient, cellulose, is weighed (or the weight verified) to an amount ranging from about 95% to about 98% of the dry weight of a composition; (2) the cellulose is added to a water, at about 10 parts cellulose to 1 part water, in stainless steel jacketed melt tanks. There are written procedures available for the weighing and manufacture of HPMC solution, and the sanitation of the equipment used; (3) a hydrogenated saccharide is weighed to about 0.5 to about 5.5% of the dry weight of the composition; (4) a lubricant is weighed to about 0.2% to about 2.5% of the dry weight of the composition; (5) the hydrogenated saccharide and lubricant are added to the cellulose and water in said stainless steel jacketed melt tanks; (b) a flavoring solution is prepared with food-grade flavor powder and cold water, or alternatively a flavored liquid base is weighed, to about 1 part flavoring to 49 parts vegetarian cellulose solution to about 1 part flavoring to 4 parts vegetarian cellulose solution by liquid weight; (c) the flavoring solution or liquid base is added to the flavorless vegetarian cellulose solution; (d) the flavored vegetarian cellulose solution is melted and de-bubbled at a temperature of about 10 degrees centigrade to about 20 degrees centigrade; (e) additional water and lubricant are added to achieve a specific gravity of about 1 gm/cm.sup.3; (f) the flavored vegetarian cellulose solution is transported to the machine that produces vegetable capsules; (g) hot molds, specifically pins, are dipped into the solution to form the capsule parts of cap and body; (h) after the flavored vegetarian cellulose solution is adhered to the mold and submitted to a drying process with heat and dry air; (i) the formed parts (cap and body) are stripped from the molds using additional lubricant; (j) the cap and body are joined.

13. The method of claim 11 further comprising the steps of: (a) preparing a dye solution by weighing dye powders and dissolving them in hot water; (b) adding the dye solution to the flavored vegetarian cellulose solution in the stainless steel tanks before it is melted and de-bubbled.

14. The method of claim 11 wherein the flavoring solution comprises a flavor chosen from the group consisting of strawberry, grape, mint, orange, blueberry, chocolate, cocoa, lemon, nut, almond, cashew, macadamia nut, coconut, blueberry, blackberry, raspberry, peach, lemon, lime, banana, chili pepper, pepper, cinnamon, and pineapple.

15. The method of claim 11 wherein said cellulose comprises a cellulose selected from the group consisting of polyacrylates, polymethacrylates, polyvinylpyrrolidone, poly(vinyl acetate), various starches, corn products such as amaizo, amylose and zein, pectin, alkoxylated celluloses, polyesters, polyethers, polyethylene glycol, proteins, nucleic acids, albumin, gelatin, starch, collagen, dextran and modified dextrans, polysaccharides, polylactide/polyglycolide, polyalkylcyanoacrylates, polyacrylamide, polysorbates, polyethylene ethers, polyethylene esters, polyoxyethylene/polyoxypropylene block polymerss, cellulose acetophthalate, hydroxypropylmethyl cellulose, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, methyl cellulose, ethyl cellulose, hydroxyethyl cellulose, propyl cellulose, hydroxypropyl cellulose, lower-substituted hydroxypropyl cellulose, carboxymethyl cellulose, and hydroxypropylmethyl cellulose.

16. The method of claim 11 wherein said cellulose is hydroxypropylmethyl cellulose.

17. The method of claim 11 wherein said hydrogenated saccharide is selected from the group consisting of sorbitol, mannitol, allitol, galactitol, L-mannitol, altritol, L-glucitol, iditol, and hexitol.

18. The method of claim 11 wherein said hydrogenated saccharide is sorbitol.

19. The method of claim 11 wherein said lubricant is selected from the group consisting of silicon dioxide, titanium dioxide, calcium phosphate, calcium sulfate , sodium laurel sulfate, silicified microcrystalline cellulose, magnesium stearate, and talc.

20. The method of claim 11 wherein said lubricant is silicon dioxide.
